(19) United States
(12) Patent Application Publication
(10) Pub. No.: US 2023/0104848 A1
(43) Pub. Date: Apr. 6, 2023

(54) INTRAVITAL COOLING DEVICE

(71) Applicant: ANT5 Co., Ltd., Yamaguchi (JP)

(72) Inventors: Michiyasu SUZUKI, Yamaguchi (JP); Takao INOUE, Yamaguchi (JP)

(21) Appl. No.: 17/949,735

(22) Filed: Sep. 21, 2022

Related U.S. Application Data
(63) Continuation of application No. PCT/JP2021/011726, filed on Mar. 22, 2021.

(30) Foreign Application Priority Data
Mar. 25, 2020 (JP) 2020-053927

(51) Int. Cl. A61F 7/12; A61F 7/00

(52) U.S. Cl. CPC A61F 7/12; A61F 7/0085; A61F 2007/126; A61F 2007/0056

(57) ABSTRACT

An intravital cooling device is for cooling a target site in a living body and includes: a heat exchange part having a flow channel through which a refrigerant passes; and a flexible heat conduction sheet that is directly or indirectly connected to the heat exchange part and arranged to cover the target site, wherein the heat conduction sheet includes a living-body surface, which is a surface on the side that makes contact with the target site, and an outer surface, which is a surface opposite from the living-body surface.

### CROSS-REFERENCE TO RELATED APPLICATION(S)

This application is a continuation application of PCT International Application No. PCT/JP2021/011726 filed on Mar. 22, 2021, which designated the United States, and which claims the benefit of priority from Japanese Patent Application No. 2020-053927, filed on Mar. 25, 2020. The entire contents of these applications are incorporated herein by reference.

### BACKGROUND OF THE INVENTION

### Field of the Invention

The present invention relates to medical intravital cooling devices for direct cooling of organs within the living body, and specifically relates to intravital cooling devices for focal brain cooling.

### Description of the Related Art

Edema or hematoma resulting from Stroke or traumatic brain injury (TBI) may result in increased brain tissue pressure or intracranial pressure, which may cause brain hypoxia due to reduction of cerebral blood flow. An example of procedure for reducing intracranial pressure includes decompressive craniotomy. Decompressive craniotomy is known as a therapeutic modality for TBI and/or stroke patients. One of the causes of serious sequelae following TBI and/or Stroke is the increase in brain temperature. In order to suppress the increase in brain temperature, as a cerebral low-temperature therapy, focal brain cooling using a device has been proposed.

JP2011-83316 A discloses a focal cooling system for suppressing refractory epileptic seizures, and/or for treating head injury and/or central nervous system diseases such as intractable pain. The system includes a cooling means for cooling a region of the brain, and performs adjustment and control of focal cooling. The focal cooling system disclosed in JP2011-83316 A includes: a cooling part that is to be embedded in a location in the body requiring cooling, the cooling part being formed by having a temperature detecting sensor attached to a bag-like container made of a flexible material or a low-profile container made of a metallic material with high heat conductivity; a coupling-connection part composed of: a catheter coupled to the cooling part and circulates cooling water; and wiring to the temperature detecting sensor; a heat radiating part that includes: a reservoir being coupled to the catheter of the coupling-connection part and in which the cooling water stays and to which a cooler is attached; and a pump that circulates the cooling water via the catheter between the reservoir and the cooling part; and a control part that is connected to the temperature detecting sensor in the cooling part via wiring and to each of the pump and the cooler in the heat radiation part via wiring, the control part controlling operation of the cooler and the pump in order to cool the location in the body requiring cooling to a predetermined temperature based on the detected temperature.

### BRIEF SUMMARY OF THE INVENTION

An intravital cooling device according to an aspect of the present invention is an intravital cooling device for cooling a target site in a living body. The device includes: a heat exchange part having a flow channel through which a refrigerant passes; and a flexible heat conduction sheet that is directly or indirectly connected to the heat exchange part and arranged to cover the target site, wherein the heat conduction sheet includes a living-body surface, which is a surface on the side that makes contact with the target site, and an outer surface, which is a surface opposite from the living-body surface.

The above-described and other features, advantages and technical and industrial significance of the present invention, will be better understood by reading the following detailed description of the current preferred embodiments of the present invention while considering the attached drawings.

### BRIEF DESCRIPTION OF THE DRAWINGS

FIG. 1 is a plan view showing an intravital cooling device according to a first embodiment of the present invention.

FIG. 2 is a cross-section view through A-A shown in FIG. 1.

FIG. 3 is a cross-section view showing a first variation of the intravital cooling device according to the first embodiment of the present invention.

FIG. 4 is a cross-section view showing a second variation of the intravital cooling device according to the first embodiment of the present invention.

FIG. 5 is a cross-section view showing a third variation of the intravital cooling device according to the first embodiment of the present invention.

FIG. 6 is a cross-section view showing an intravital cooling device according to a second embodiment of the present invention.

FIG. 7 is a cross-section view showing a first variation of the intravital cooling device according to the second embodiment of the present invention.

FIG. 8 is a cross-section view showing a second variation of the intravital cooling device according to the second embodiment of the present invention.

FIG. 9 is a plan view showing an intravital cooling device according to a third embodiment of the present invention.

FIG. 10 is a plan view showing an intravital cooling device according to a fourth embodiment of the present invention.

FIG. 11A is a schematic diagram illustrating a cross-section through B-B shown in FIG. 10.

FIG. 11B is a schematic diagram illustrating a cross-section through B-B shown in FIG. 10.

FIG. 11C is a schematic diagram illustrating a cross-section through B-B shown in FIG. 10.

FIG. 12 is a cross-section view showing a variation of the intravital cooling device according to the fourth embodiment of the present invention.

### DETAILED DESCRIPTION OF THE INVENTION

Hereinafter, an intravital cooling device according to embodiments of the present invention will be described with reference to the drawings. It should be noted that the present invention is not limited by these embodiments. In the description of each drawing, the same parts are denoted by the same reference numbers.

The drawings referred to in the following description are merely schematic representations of shape, size, and positional relationship to the extent that the subject matter of the present invention may be understood. In other words, the present invention is not limited only to the shapes, sizes, and positional relationships illustrated in the respective figures. In addition, the drawings may also include, among themselves, parts having different dimensional relationships and ratios from each other.

The intravital cooling device according to each embodiment of the present invention is a device that is retained in the patient's living body for a certain period of time to focally cool organs such as a brain. Such device is used in conjunction with an intravital cooling system provided with various sensors, controllers, and the like. The intravital cooling device may preferably be used as a device for cooling the brain surface section in the cerebral low-temperature therapy, which is applied to patients with traumatic cerebral contusion, or the like, in the acute phase or perioperative phase, or in treatment for suppressing epileptic seizures. In the cerebral low-temperature therapy, a sensor is placed on the brain surface after dura mater dissection, the dura mater is placed back, and an intravital cooling device is placed on such dura mater. Then, depending on the measurement results of parameters, such as temperature, by the sensor, the parameters, such as the temperature of a refrigerant supplied to a heat exchange part (described below) of the intravital cooling device, the supply speed (flow rate) of the refrigerant, the cooling time, and the like, are controlled. In cerebral low-temperature therapy, the intravital cooling device may be retained for, for example, one week. It should be noted that, as for the intravital cooling device according to the present invention, devices that cool the interior of the living body from the body surface, i.e., from outside of the body, are excluded.

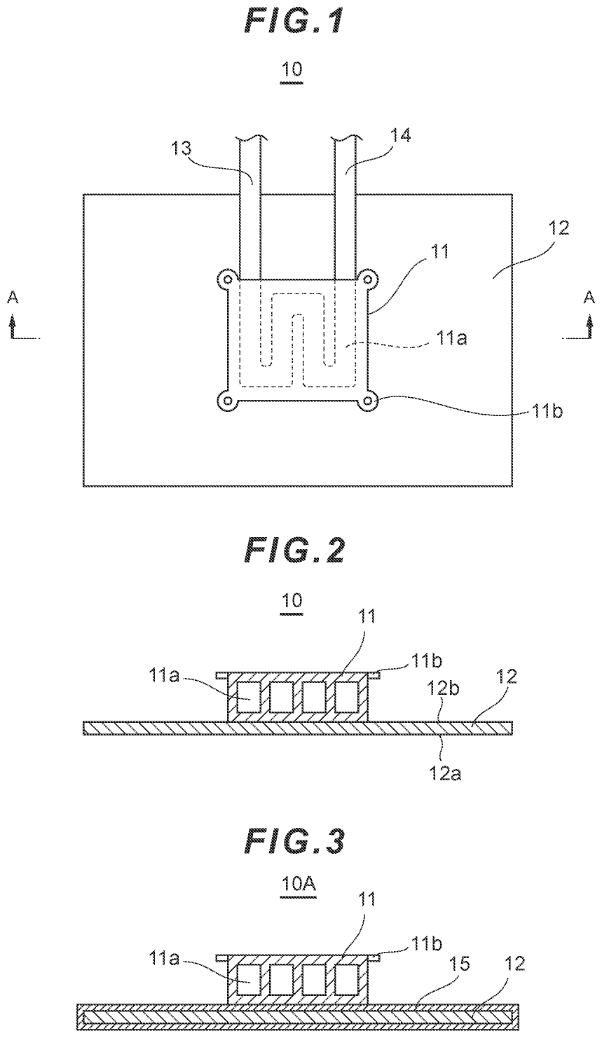

FIG. 1 is a plan view showing an intravital cooling device (which may also be referred to hereinafter simply as a “cooling device”) according to a first embodiment of the present invention. FIG. 2 is a cross-section view through A-A shown in FIG. 1. As shown in FIGS. 1 and 2, the cooling device 10 according to the present embodiment is a device for cooling a target site (a site to be treated) in the living body. The device includes: a heat exchange part 11 having a flow channel 11a through which a refrigerant passes; and a heat conduction sheet 12 which is directly or indirectly connected to the heat exchange part 11 and arranged to cover the site to be treated. The heat conduction sheet 12 includes: a living-body surface 12a, which is a surface on the side that makes contact with the target site; and an outer surface 12b, which is a surface opposite from the living-body surface 12a.

As shown in FIGS. 1 and 2, the heat exchange part 11 is arranged on the outer surface 12b side of the heat conduction sheet 12. The position of the heat exchange part 11 on the outer surface 12b is not particularly limited. In terms of increasing the contact area between the heat exchange part 11 and the heat conduction sheet 12 and the miniaturization of the cooling device 10, it is preferable to arrange the cooling device 10 inside the outer circumference of the heat conduction sheet 12. However, arranging a portion of the heat exchange part 11 outside the outer circumference of the heat conduction sheet 12 is not excluded. In terms of cooling the entire heat conduction sheet 12 quickly, it is preferable to arrange the heat exchange part 11 in approximately the center of the heat conduction sheet 12.

The heat exchange part 11 performs heat exchange with the heat conduction sheet 12 by allowing the refrigerant to flow through the internally formed flow channel 11a. The types of refrigerant are not particularly limited; however, for example, Ringer's solution, saline, or pure water may be used. When the cooling device 10 is used in cerebral low-temperature therapy, if the brain surface section is to be cooled to approximately 15 to 25 degrees, the temperature of the refrigerant to flow through the flow channel 11a may be set, for example, to 1 degree or more, preferably 3 degrees or more, and more preferably 5 degrees or more, and for example, 20 degrees or less, preferably 15 degrees or less, and more preferably 10 degrees or less. The flow rate of the refrigerant may be set, for example, to 400 mL/min or more.

An inflow pipe 13 for allowing the refrigerant to flow into the flow channel 11a and an outflow pipe 14 for allowing the refrigerant to flow out of the flow channel 11a are connected to the heat exchange part 11. The ends of the inflow pipe 13 and the outflow pipe 14, opposite from the heat exchange part 11, are connected to, for example, a refrigerant circulation part for cooling and circulating the refrigerant. The refrigerant circulation part includes, for example, a tank in which the refrigerant is stored, a cooler for cooling the refrigerant in the tank, and a pump for circulating the refrigerant among the tank, the inflow pipe 13, and the outflow pipe 14. It should be noted that in FIG. 1, the inflow pipe 13 and the outflow pipe 14 are connected to the side surface of the heat exchange part 11; however, the positions where the inflow pipe 13 and the outflow pipe 14 are connected are not limited thereto. For example, the inflow pipe 13 and the outflow pipe 14 may be connected to the top surface of the heat exchange part 11.

The planar shape of the flow channel 11a is not particularly limited; however, it is preferable to have a shape such that the refrigerant easily flows from the inlet to the flow channel 11a toward the outlet and the refrigerant is unlikely to stagnate within the flow channel 11a. Examples of the planar shape of the flow channel 11a may include a meander shape, as shown in FIG. 1, a spiral shape, or a simple straight line. In addition, the width of the flow channel 11a does not necessarily have to be constant. Further, the flow channel 11a may have a shape in which the flow channel 11a diverges into multiple channels or merges from multiple channels within the heat exchange part 11. Alternatively, multiple channels may be provided in the heat exchange part 11.

In the heat exchange part 11 shown in FIG. 1, the flow channel 11a is formed inside a solid body, but the heat exchange part may be formed by, for example, arranging a tubular flow channel inside a box-like container. In short, the configuration is sufficient if heat exchange can be achieved between the refrigerant flowing inside the flow channel 11a and the heat conduction sheet 12 that makes contact with the bottom surface of the heat exchange part 11. In addition, the outer shape of the heat exchange part 11 is not particularly limited, and it may be rectangular, as shown in FIG. 1, or columnar, such as prismatic or cylindrical, frustum, tubular, and so on.

The size of the heat exchange part 11 is not particularly limited as long as it can be retained in the living body, but may be set appropriately depending on conditions, such as the position and size of the site to be treated, the size of the heat conduction sheet 12, and the like. As an example, if the planar shape of the heat exchange part 11 is rectangular as shown in FIG. 1, the length of one side in the plane of the heat exchange part 11 may be approximately 1 cm to 7 cm.

A fixing protrusion 11b may be provided on the outer surface (top or side surface) of the heat exchange part 11 in order to fix the heat exchange part 11 to the living body. The fixing protrusion 11b may be used to fix the heat exchange part 11 by, for example, sewing it on the inside of the scalp. The fixing protrusion 11b shown in FIG. 1 has a shape in which a through-hole is formed in the center of the substantially disc-shaped protrusion, but various shapes may be employed for the fixing protrusion 11b, including a partially indented shape, a hook-like shape, and the like.

Such heat exchange part 11 may be formed using, for example: metals such as titanium, copper, and silver; stainless steels such as SUS301, SUS303, SUS304, and SUS631; and/or alloys such as Ni-Ti alloy, and aluminum alloy. Alternatively, synthetic resins such as polyolefin-based resins including polypropylene, fluorine-based resins including tetrafluoroethylene, or silicone-based resin, synthetic rubber such as ethylene propylene diene rubber, and natural rubber, may be used to form the heat exchange part 11. Alternatively, a plurality of different materials, such as metals and synthetic resin materials, may be used to form the heat exchange part 11. In this case, it is preferable to use materials with relatively high heat conductivity, such as metals and alloys, for at least the portion of the heat exchange part 11 that is connected to the heat conduction sheet 12. In addition, flexible materials may be used to form the heat exchange part 11. This enables the heat exchange part 11 to be arranged alongside the site to be treated. Further, the surface of the heat exchange part 11 may be coated with a biocompatible material such as parylene (registered trademark).

The heat conduction sheet 12 is a flexible sheet-like member and is provided in order to transfer heat generated at the site to be treated to the refrigerant flowing within the heat exchange part 11. Here, the “sheet” referred to herein is not limited in terms of its thickness and may include a laminate-like member, a paper-like member, a membrane-like member, a thin-membrane-like member, or a film-like member.

The heat conductivity of the heat conduction sheet 12 is at least higher than the heat conductivity of the living body tissue, and preferably equal to or higher than the heat conductivity of the portion of the heat exchange part 11 that is connected to the heat conduction sheet 12. Here, the heat conductivity of the living body tissue varies depending on the tissue, but it is generally considered to be approximately 0.5 W/mK. As an example, if the heat exchange part 11 is made of titanium (heat conductivity: approximately 17 W/mK), it is preferable for the heat conduction sheet 12 to have heat conductivity of approximately 17 W/mK or higher. Thus, by defining the heat conductivity of the heat conduction sheet 12, it is possible to conduct heat quickly over a wide range of the heat conduction sheet 12 covering the living body and to efficiently exchange heat with the refrigerant flowing in the heat exchange part 11. Of course, the heat conductivity of the heat conduction sheet 12 may be a few tens of W/mK or higher, or a few hundreds of W/mK or higher.

The materials of the heat conduction sheet 12 are not particularly limited as long as they are flexible and capable of achieving sufficient heat conductivity. Specific examples may include: metal foils such as gold, silver, copper, and titanium; graphite sheets; and sheets formed by high heat conductive resins. Graphite sheets in particular have very high heat conductivity (for example, several hundred to 1,000 W/mK or higher) and are preferred as materials for the heat conduction sheet 12. Here, the basic structure of a graphite crystal is a layered structure in which the basal planes formed by hexagonal net-like linked carbon atoms are regularly stacked (the direction in which the layers are stacked is referred to as the “c-axis” and the direction in which the basal planes formed by the hexagonal net-like linked carbon atoms extend is referred to as the “Basal plane (a-b plane) direction”). The carbon atoms in the basal planes are tightly linked through covalent binding, whereas the stacked layer surfaces are bound through van der Waals force, which is weak. Therefore, the graphite sheet reflects such anisotropy and has a large heat conductivity in the plane direction (a-b plane direction). This allows preferential heat diffusion in the plane direction of the heat conduction sheet 12, thereby effectively cooling a wide range of the site to be treated in the living body. When a graphite sheet is used as the heat conduction sheet 12, a sheet laminated with PET, polyimide, or the like on one or both sides of graphite may be used.

The thickness of the heat conduction sheet 12 is not particularly limited as long as flexibility is ensured to the extent that the heat conduction sheet 12 can generally be closely fitted to the site to be treated and tearing or splitting of the heat conduction sheet 12 can be suppressed. For example, if the heat conduction sheet 12 is to be formed by graphite sheets, the thickness is preferably 1000 μm or less, more preferably 800 μm or less, even more preferably 600 μm or less, and particularly preferably 400 μm or less, in order to ensure flexibility to allow easy close-fitting to the site to be treated. In addition, the thickness is preferably 30 μm or more, more preferably 100 μm or more, and even more preferably 200 μm or more, in order to ensure heat capacity while preventing tearing or splitting of the heat conduction sheet 12. Even when materials other than graphite sheets are used as the material for the heat conduction sheet 12, the thickness can be determined accordingly, taking into consideration the property of close-fitting to the site to be treated, the prevention of breakage, and the like.

The planar shape of the heat conduction sheet 12 is not particularly limited as long as it can cover the site to be treated with the living-body surface 12a. For example, the planar shape of the heat conduction sheet 12 may be rectangular, as shown in FIG. 1, circular, elliptical, polygonal, or a shape formed by combining these shapes. In addition, through-holes, protrusions, notches, or the like, may be formed in a portion of the heat conduction sheet 12 for fixing the heat conduction sheet 12 by, for example, sewing it on the dura mater.

When using such a cooling device 10, the heat conduction sheet 12 is arranged on the dura mater covering the site to be treated, and the heat exchange part 11 is arranged inside the scalp. At this time, the heat conduction sheet 12 may be fixed by, for example, sewing it onto the dura mater. The heat exchange part 11 may be fixed to the scalp using the fixing protrusions 11b. Then, the heat exchange part 11 is connected to the heat conduction sheet 12 by placing the scalp over the dura mater. In this state, the site to be treated is cooled by supplying the refrigerant to the heat exchange part 11 and allowing it to circulate in the flow channel 11a. At this time, depending on the measurement results of parameters, such as temperature, by the sensor, which is pre-arranged on the brain surface, the temperature of the refrigerant supplied to the heat exchange part 11, the supply speed (flow rate) of the refrigerant, the cooling time, and the like, may be preferably controlled.

Here, in a conventional cooling device in which heat exchange is achieved in a cooling container through inflow and outflow of cooling water into/from the cooling container, it is difficult to control the flow of cooling water locally within the cooling container, and this may therefore reduce the efficiency of the circulation of cooling water in the cooling container. This may result in temperature variations at the surface of the cooling container to be brought close to a site to be treated. When the low-profile metal container is used as the cooling container, it is difficult to cover a wide range of the site to be treated with irregularities using such low-profile metal container, which is rigid. Therefore, a cooling device that can efficiently cool a wide range of the site to be treated has been desired. In addition, the weight and thickness of the cooling container may become a burden on the patients if the cooling container filled with cooling water is brought close to the site to be treated. Therefore, the improvement has been also desired from the perspective that the device will be retained in the living body for a period of time.

According to the present embodiment, the flexible heat conduction sheet is connected directly or indirectly to the heat exchange part having the flow channel through which the refrigerant passes. Therefore, the heat conduction sheet can be flexibly deformed to cover a wide portion of the target site in the living body, thereby enhancing the close-fitting property of the heat conduction sheet to the relevant site. In addition, the heat exchange part has the flow channel. Therefore, the refrigerant can be circulated efficiently in the heat exchange part. Accordingly, it is possible to realize an intravital cooling device that is easy to retain in the living body and that can effectively cool a wide range of the site to be treated.

In detail, according to the first embodiment of the present invention, the flow channel 11a is provided in the heat exchange part 11, and the stagnation of refrigerant in the heat exchange part 11 can therefore be suppressed and the refrigerant may therefore be allowed to circulate efficiently. Accordingly, the cooling efficiency of the cooling device 10 can be improved.

In addition, according to the first embodiment of the present invention, the flexible heat conduction sheet 12 is used, and a wide portion of the site to be treated can therefore be covered and the property of close-fitting to such site can therefore be enhanced by flexibly deforming the heat conduction sheet 12 according to the shape of the site to be treated. In addition, the heat conductivity of the heat conduction sheet 12 is preferably equal to or higher than the heat conductivity of the portion of the heat exchange part 11 that is connected to the heat conduction sheet 12. Therefore, it is possible to conduct heat quickly in the heat conduction sheet 12 and to efficiently exchange heat with the refrigerant flowing in the heat exchange part 11. This allows for a wider range, with respect to the size of the heat exchange part 11, to be cooled via the heat conduction sheet 12. Accordingly, it is possible to realize a cooling device that is easy to retain in the living body and that can effectively cool a wide range of the site to be treated.

In addition, according to the first embodiment of the present invention, the size of the heat exchange part 11 can be made smaller with respect to the heat conduction sheet 12 covering the site to be treated, and the burden on a patient to whom the cooling device 10 is applied can therefore be reduced.

It should be noted that, in the above-described first embodiment, the heat exchange part 11 is fixed to the scalp and the heat conduction sheet 12 is fixed on the dura mater, and they are then connected to each other; however, the heat conduction sheet 12 may be fixed to the heat exchange part 11 in advance by using a mechanical fixing means such as a clip. The fixing means such as a clip may be either integral with or separate from the heat exchange part 11. In this case, the cooling device can be used by fixing either the heat exchange part 11 or the heat conduction sheet 12 in the living body.

FIG. 3 is a cross-section view showing a first variation of the cooling device 10 according to the first embodiment of the present invention. The cooling device 10A shown in FIG. 3 corresponds to the cooling device 10 shown in FIG. 2 having a coating film 15 formed on the surface of the heat conduction sheet 12. The coating film 15 is made of a biocompatible material such as parylene (registered trademark), and it may be formed by, for example, sputtering or vacuum deposition. The thickness of the coating film 15 is preferably a few tens of μm or less in order to ensure sufficient heat exchange efficiency with respect to the heat exchange part 11 and the living body and to ensure the flexibility of the heat conduction sheet 12. In addition, the thickness of the coating film 15 is preferably several hundreds of nm or more, and more preferably several μm or more, in order to prevent breakage such as shaving or peeling. As an example, the thickness of the coating film 15 may be between approximately 10 μm or more and approximately 20 μm or less (i.e., a dozen μm).

By providing such coating film 15, it is possible to add biocompatibility to the heat conduction sheet 12 and thereby increase the range of material selection for the heat conduction sheet 12. The coating film 15 also allows for an improvement in the durability of the heat conduction sheet 12.

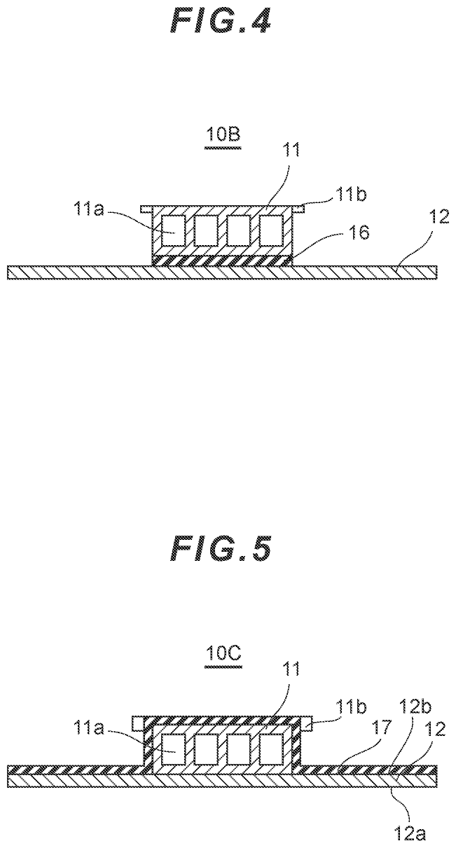

FIG. 4 is a cross-section view showing a second variation of the cooling device 10 according to the first embodiment of the present invention. The cooling device 10B shown in FIG. 4 corresponds to the cooling device 10 shown in FIG. 2 further having an intermediate layer 16 arranged between the heat exchange part 11 and the heat conduction sheet 12. It should be noted that the heat conduction sheet 12 formed with the coating film 15 (see FIG. 3) may be used instead of the heat conduction sheet 12 shown in FIG. 4.

The intermediate layer 16 is made of an adhesive material and is provided in order to improve the close-fitting property between the heat exchange part 11 and the heat conduction sheet 12. The state of the intermediate layer 16 is not particularly limited, and it may be in, for example, a gel form, paste form, or sheet form such as a gel sheet or adhesive sheet. The intermediate layer 16 may also be a paste (adhesive) which cures under certain conditions. In this case, the adhesive property may not need to remain in the intermediate layer 16 after curing. Specifically, epoxy resin-based adhesives, acrylic resin-based adhesives, cyanoacrylate-based adhesives, silicone resin-based adhesives, silicone gel sheets, double-sided adhesive films, heat-adhesive films, thermal compression bonded films, and the like, may be used as the intermediate layer 16.

As a material for the intermediate layer 16, it is preferable for it to be a material with biocompatibility such as silicone, and is also preferable for it to be a material with good heat conductivity. For example, the intermediate layer 16 may be a material having a heat conductive filler added to the base material such as a silicone gel.

If the intermediate layer 16 is provided, the heat exchange part 11 and the heat conduction sheet 12 may be integrated together in advance. In this case, the cooling device 10B can be used with either the heat exchange part 11 or the heat conduction sheet 12 being fixed in the living body. For example, if the heat conduction sheet 12 is to be fixed in the living body (e.g., on the dura mater), the fixing protrusion 11b may be omitted since the heat exchange part 11 does not need to be fixed to the scalp.

Alternatively, the intermediate layer 16 may be arranged on a side of at least one of the heat exchange part 11 or the heat conduction sheet 12, and the heat exchange part 11 and the heat conduction sheet 12 may be integrated together when the cooling device 10B is used as in the above-described first embodiment.

According to the second variation of the cooling device according to the first embodiment, the close-fitting property between the heat exchange part 11 and the heat conduction sheet 12 can be improved by the intermediate layer 16, and the heat exchange efficiency between the two can therefore be improved. In addition, the heat exchange part 11 can be prevented from making direct contact with the heat conduction sheet 12, and an effect of protecting the outer surface 12b of the heat conduction sheet 12 may therefore be obtained. Further, the heat exchange part 11 and the heat conduction sheet 12 can be integrated together without using a mechanical fixing means such as a clip, and unexpected breakage of the heat conduction sheet 12 can therefore be prevented.

FIG. 5 is a cross-section view showing a third variation of the cooling device 10 according to the first embodiment of the present invention. The cooling device 10C shown in FIG. 5 corresponds to the cooling device 10 shown in FIG. 2 further having a heat-insulating layer 17 arranged on the heat exchange part 11 and on a region on the outer surface 12b side of the heat conduction sheet 12.

The heat-insulating layer 17 is formed by a heat-insulating sheet, a heat-insulating film, or the like, having heat conductivity lower than that of the heat conduction sheet 12, and is provided in order to prevent the outer surface side of the cooling device 10C from becoming too cold. The heat-insulating layer 17 may be provided throughout the region on the outer surface side of the heat exchange part 11 and the heat conduction sheet 12 (see FIG. 5), or it may be partially provided such as: only on the surface of the heat exchange part 11; only on the outer surface 12b of the heat conduction sheet 12; or only on part of the outer surface 12b around the heat exchange part 11. If the heat-insulating layer 17 is to be provided integrally throughout the region on the outer surface side of the heat exchange part 11 and the heat conduction sheet 12, the heat exchange part 11 and the heat conduction sheet 12 can be integrated together.

The heat-insulating layer 17 may contain a base material and bubbles or fillers dispersed in the base material. By dispersing, in the base material, bubbles or fillers with a higher specific heat capacity or lower heat conductivity than the base material, heat may be reflected or absorbed in the heat-insulating layer 17. The heat-insulating layer 17 may have a sea-island structure where the base material corresponds to the sea and the bubbles or fillers correspond to the islands.

Resin may be used as the base material for the heat-insulating layer 17. Specific examples include polyolefin-based resins such as polyethylene and polypropylene, polyamide-based resins, polyester-based resins, polyurethane-based resins, polyimide-based resins, fluorine-based resins, vinyl chloride-based resins, silicone-based resins, natural rubbers, synthetic rubbers, and the like.

If bubbles are present in the heat-insulating layer 17, it is preferable for the heat-insulating layer 17 to have a closed cell structure because of its superior heat insulation properties. The type of gas contained in the bubbles is not particularly limited, and, for example, air or nitrogen may be used.

The shape of the fillers is not particularly limited, but it can be: granular such as spherical; needle-like; fibrous; or plate-like. The fillers can be in either a solid form or a hollow form, but it is preferable to be in a hollow form for lightweight and high heat insulation effect. The materials that make up the filler are not particularly limited, and can be organic or inorganic materials, or organic-inorganic composite materials. Examples of organic materials include: thermosetting resins such as phenol, epoxy, and urea; or thermoplastic resins such as polyester, polyvinylidene chloride, polystyrene, and polymethacrylate. Examples of inorganic materials include shirasu, pearlite, glass, silica, alumina, zirconia, and carbon.

The method of arranging the heat-insulating layer 17 on the heat exchange part 11 and the heat conduction sheet 12 is not particularly limited. For example, an adhesive or adhesive sheet may be used to attach the heat-insulating layer 17 to the heat exchange part 11 and the heat conduction sheet 12.

According to the third variation of the cooling device according to the first embodiment, the heat-insulating layer 17 is provided, and the outer surface side of the cooling device 10C can therefore be prevented from becoming too cold. This allows for the suppression of cooling of unscheduled sections in the living body and the enhancement of the cooling effect to the site to be treated. In addition, the temperature distribution in the heat conduction sheet 12 can be easily achieved, and the entire site to be treated can therefore be cooled in a uniform manner.

In FIG. 5, the heat exchange part 11 and the heat conduction sheet 12 are directly connected to each other. However, as in the above-described first variation (see FIG. 3), a coating film 15 may be formed on the surface of the heat conduction sheet 12, or, as in the above-described second variation (see FIG. 4), an intermediate layer 16 may be interposed between the heat exchange part 11 and the heat conduction sheet 12. In the case of interposing the intermediate layer 16, the intermediate layer 16 can be used as a means of adhesion between the heat conduction sheet 12 and the heat-insulating layer 17 by arranging the intermediate layer 16 over the entire outer surface 12b of the heat conduction sheet 12.

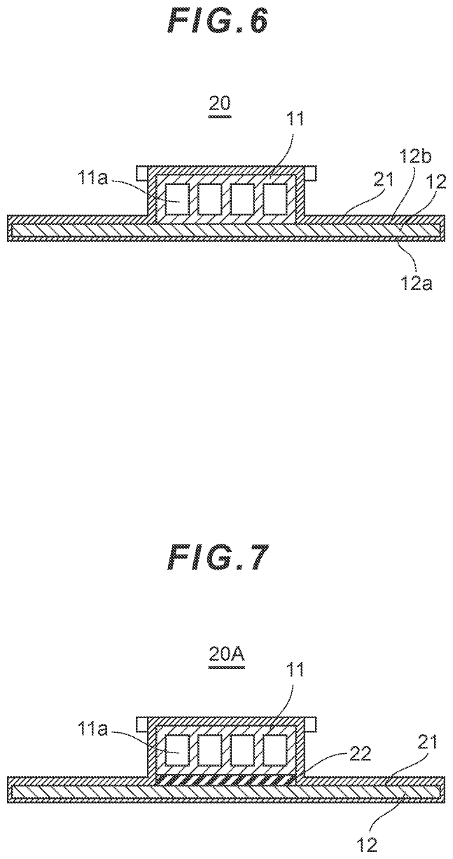

Next, a second embodiment of the present invention will be described. FIG. 6 is a cross-section view showing a cooling device according to the second embodiment of the present invention. The cooling device 20 according to the present embodiment includes the heat exchange part 11, the heat conduction sheet 12 which is connected directly or indirectly to the heat exchange part 11, and a covering layer 21 which integrally covers the heat exchange part 11 and the heat conduction sheet 12. The configuration and function of the heat exchange part 11 and the heat conduction sheet 12 are similar to those of the above-described first embodiment.

The covering layer 21 is made of a biocompatible material such as parylene (registered trademark), and is provided in order to provide biocompatibility to the heat exchange part 11 and the heat conduction sheet 12 and to integrate both. Such covering layer 21 may be formed by, for example, sputtering or vacuum deposition.

The thickness of the covering layer 21 is preferably a few tens of μm or less in order to ensure sufficient heat exchange efficiency with respect to the living body and to ensure the flexibility of the heat conduction sheet 12. In addition, the thickness of the covering layer 21 is preferably several hundreds of nm or more, and more preferably several μm or more, in order to prevent breakage such as shaving or peeling. As an example, the thickness of the covering layer 21 may be between approximately 10 μm or more and approximately 20 μm or less (i.e., a dozen μm).

The thickness of the covering layer 21 may be generally uniform or partially varied across the entire surface of the heat exchange part 11 and the heat conduction sheet 12. For example, as shown in FIG. 6, the thickness of the portion of the covering layer 21 covering the heat exchange part 11 and the outer surface 12b of the heat conduction sheet 12 may be increased compared with that of the portion covering the living-body surface 12a of the heat conduction sheet 12. In this case, the reduction in heat exchange efficiency between the heat conduction sheet 12 and the living body may be suppressed on the living-body surface 12a side, and the heat exchange part 11 and the heat conduction sheet 12 may be integrated together with sufficient strength on the outer surface 12b side. As an example, the thickness of the covering layer 21 on the living-body surface 12a side may be between approximately several hundreds of nm and approximately a dozen μm, and the covering layer 21 on the outer surface 12b side may be between approximately a dozen μm and approximately a several tens of μm.

According to the second embodiment of the present invention, the heat exchange part 11 and the heat conduction sheet 12 can be integrated together by means of the covering layer 21 without using adhesives or a mechanical fixing means. In addition, biocompatibility may be added to the cooling device 20, thereby increasing the range of material selection for each part configuring the cooling device 20.

FIG. 7 is a cross-section view showing a first variation of the cooling device 20 according to the second embodiment of the present invention. The cooling device 20A shown in FIG. 7 corresponds to the cooling device 20 shown in FIG. 6 further having an intermediate layer 22 arranged between the heat exchange part 11 and the heat conduction sheet 12. The intermediate layer 22 is made of an adhesive material and is provided in order to improve the close-fitting property between the heat exchange part 11 and the heat conduction sheet 12. The state of the intermediate layer 22 is preferably in a sheet form such as a gel sheet or adhesive sheet. In addition, the intermediate layer 22 may be a paste (adhesive) which cures under certain conditions, and in this case, the adhesive property may not need to remain in the intermediate layer 22 after curing.

As a material for the intermediate layer 22, it is preferable for it to be a material with good heat conductivity in terms of heat conductivity. For example, the intermediate layer 22 may be a material having a heat conductive filler added to the base material such as a silicone gel.

Here, when forming the covering layer 21 by sputtering or vacuum deposition, if a void is present between the heat exchange part 11 and the heat conduction sheet 12, a vacuum may result in such void, and there is therefore a risk that the heat exchange efficiency at the boundary between the heat exchange 11 and the heat conduction sheet 12 is reduced. Therefore, the small void between the heat exchange part 11 and the heat conduction sheet 12 may be closed by arranging the intermediate layer 22 between the heat exchange part 11 and the heat conduction sheet 12. This allows for the prevention of the generation of a vacuum at the boundary between the heat exchange part 11 and the heat conduction sheet 12 and the suppression of the reduction in heat exchange efficiency.

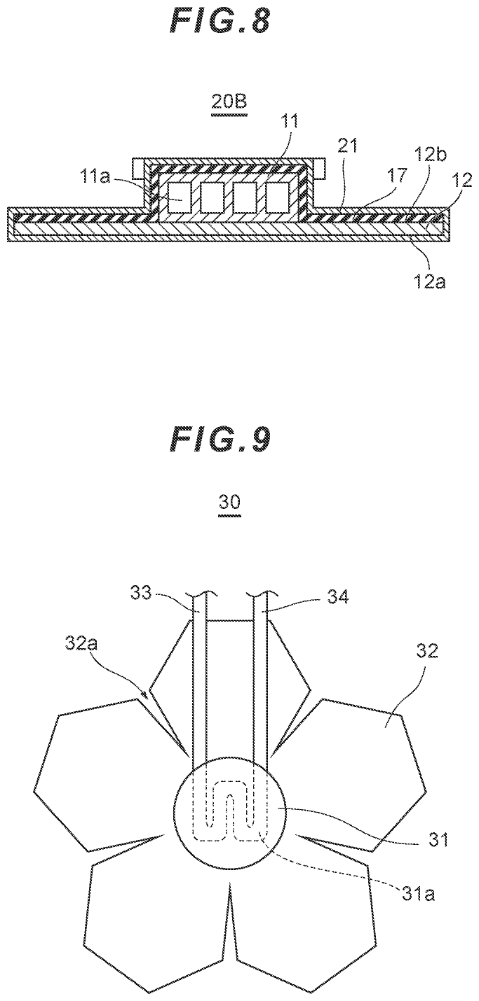

FIG. 8 is a cross-section view showing a second variation of the cooling device 20 according to the second embodiment of the present invention. The cooling device 20B shown in FIG. 8 includes: the heat-insulating layer 17 arranged on the heat exchange part 11 and the outer surface 12b side of the heat conduction sheet 12; and the covering layer 21 integrally covering the heat exchange part 11, the heat conduction sheet 12, and the heat-insulating layer 17. The configuration and function of the heat-insulating layer 17 are similar to those described in the third variation (see FIG. 5) of the first embodiment. According to the present variation, the heat-insulating layer 17 is provided, and the outer surface side of the cooling device 20B may therefore be prevented from becoming too cold. In addition, the covering layer 21 is formed on the surface of the heat-insulating layer 17, thereby increasing the range of material selection for the heat-insulating layer 17.

For the cooling device 20B shown in FIG. 8, an intermediate layer 16 (see FIG. 4) may be interposed between the heat exchange part 11 and the heat conduction sheet 12. As a further variation, after integrally covering the heat exchange part 11 and the heat conduction sheet 12 by the covering layer 21 (see FIG. 6), the heat-insulating layer 17 may be provided on the covering layer 21 on the outer surface 12b side. Breakage (shaving, peeling, etc.) of the covering layer 21 covering the heat exchange part 11 and the outer surface 12b side of the heat conduction sheet 12 may be prevented by providing the heat-insulating layer 17 on the covering layer 21.

Next, a third embodiment of the present invention will be described. FIG. 9 is a plan view showing a cooling device according to the third embodiment of the present invention. As shown in FIG. 9, the cooling device 30 according to the present embodiment includes: a heat exchange part 31 having a flow channel 31a through which a refrigerant passes; and a heat conduction sheet 32 which is connected directly or indirectly to the heat exchange part 31. An inflow pipe 33 for allowing the refrigerant to flow into the flow channel 31a and an outflow pipe 34 for allowing the refrigerant to flow out of the flow channel 31a are connected to the heat exchange part 31.

The basic configuration and function of the heat exchange part 31 and the heat conduction sheet 32 in the present embodiment are similar to those of the heat exchange part 11 and the heat conduction sheet 12 in the above-described first embodiment, except that their planar shapes are different. In particular, in the heat conduction sheet 32 in the present embodiment, one or more (five in FIG. 9) notches 32a is/are formed at the periphery. By forming such a notch 32a, the heat conduction sheet 32 may be easily deformed along the curved surface, thereby allowing the heat conduction sheet 32 to be more closely fitted to the site to be treated and thus allowing efficient cooling.

The position, number, shape, orientation, and depth of the notch 32a are not particularly limited. The position, number, and the like of notches 32a may be determined so that the heat conduction sheet 32 can be deformed according to the position, size, and three-dimensional shape of the site to be treated, and the property of close-fitting to the site to be treated can be enhanced. For example, a plurality of notches 32a that extend from the outer circumference of the heat conduction sheet 32 to its center may be arranged at regular intervals (see FIG. 9), or with different intervals. The shape of the notch 32a may also be linear, cuneiform, curved, zigzag, meandrous, or the like.

For the cooling device 30 according to the third embodiment, as with the first embodiment, a fixing protrusion 11b (see FIG. 1) may be provided to the heat exchange part 31, and/or a through-hole, protrusion, or notch for fixing the heat conduction sheet 32 in the living body may be provided in the heat conduction sheet 32. In addition, a coating film 15 (see FIG. 3) may be formed for the heat conduction sheet 32, an intermediate layer 16 (see FIG. 4) may be arranged between the heat exchange part 31 and the heat conduction sheet 32, and/or a heat-insulating layer 17 (see FIG. 5) may be added. Further, as with the second embodiment, the heat exchange part 31 and the heat conduction sheet 32 may be integrally covered by a covering layer 21 (see FIG. 6).

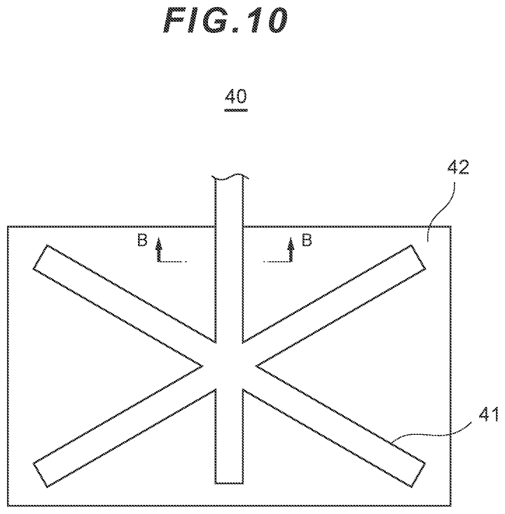

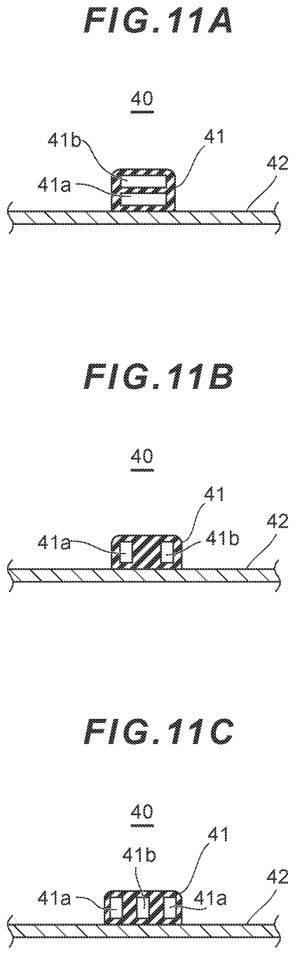

Next, a fourth embodiment of the present invention will be described. FIG. 10 is a plan view showing a cooling device according to the fourth embodiment of the present invention. FIGS. 11A to 11C are schematic diagrams illustrating cross-sections through B-B in FIG. 10. The cooling device 40 according to the present embodiment includes: a heat exchange part 41 having flow channels 41a, 41b through which refrigerants pass; and a heat conduction sheet 42 which is connected directly or indirectly to the heat exchange part 41. The configuration and function of the heat conduction sheet 42 are similar to those of the heat conduction sheet 12 in the above-described first embodiment.

As shown in FIG. 10, the heat exchange part 41 in the present embodiment is tubular. The planar shape of the heat exchange part 41 is not particularly limited, and it may, for example, have a shape in which divergent tubes radially spread out as shown in FIG. 10, or a shape with a single path.

The form of the flow channel in the transverse plane (the cross section perpendicular to the longitudinal direction of the tube) of the heat exchange part 41 is not particularly limited. For example, as shown in FIGS. 11A to 11C, the flow channel (inflow channel 41a) for allowing the refrigerant that has entered the heat exchange part 41 to pass therethrough and the flow channel (outflow channel 41b) for allowing the refrigerant to be discharged from the heat exchange part 41 to pass therethrough may be separated from each other within the heat exchange part 41. In this case, the refrigerant may be circulated by allowing the inflow channel 41a and the outflow channel 41b to communicate with each other at the ends of the divergent tubes of the heat exchange part 41 as shown in FIG. 10. In terms of the arrangement of flow channels, the inflow channel 41a and the outflow channel 41b may be stacked on top of each other as shown in FIG. 11A, or the inflow channel 41a and the outflow channel 41b may be arranged next to each other in the same plane as shown in FIG. 11B. Alternatively, as shown in FIG. 11C, the inflow channels 41a may be arranged on the outer side of the heat exchange part 41, and the outflow channel 41b may be arranged on the inner side of the heat exchange part 41. Of course, a single flow channel may be formed in the heat exchange part 41.

The cross-sectional outline of the heat exchange part 41 is not particularly limited, and may be substantially rectangular, as shown in FIGS. 11A to 11C, circular, elliptical, semicircular, polygonal, or a shape formed by combining these shapes. Considering the pressure drop of the refrigerant in the flow channels 41a, 41b and the property of close-fitting to the heat conduction sheet 42, it is preferable for the cross-sectional shape of the heat exchange part 41 to be semi-circular or polygonal including substantially rectangular. In addition, it is also preferable to connect the longer portion of the outer circumference of the cross-section of the heat exchange part 41 to the heat conduction sheet 42 in terms of increasing the contact area between the heat exchange part 41 and the heat conduction sheet 42.

The heat exchange part 41 may be made of metals or alloys, or of flexible materials. In the latter case, the heat exchange part 41 can be deformed along with the heat conduction sheet 42 to be closely fitted to the site to be treated. For example, heat exchange tubes made of silicones, rubbers such as synthetic rubbers, and fluorine-based resins such as PFA, may be used as the heat exchange part 41. In addition, materials with added heat conductive fillers may preferably be used in terms of heat conductivity.

According to the fourth embodiment of the present invention, the heat exchange part 41 is tubular, and the heat exchange part 41 may therefore be arranged on a wide range of the heat conduction sheet 42, while taking advantage of the flexibility of the heat conduction sheet 42. This allows for direct heat exchange with the heat exchange part 41 over a wide range of the heat conduction sheet 42 to efficiently cool a wide range of the site to be treated. In addition, the degree of freedom may be increased in terms of the arrangement and/or extension direction of the heat exchange part 41 with respect to the heat conduction sheet 42.

For the cooling device 40 according to the fourth embodiment, as with the first embodiment, a fixing protrusion 11b (see FIG. 1) may be provided to the heat exchange part 41, and/or a through-hole, protrusion, or notch for fixing the heat conduction sheet 42 in the living body may be provided in the heat conduction sheet 42. In addition, a coating film 15 (see FIG. 3) may be formed for the heat conduction sheet 42, an intermediate layer 16 (see FIG. 4) may be arranged between the heat exchange part 41 and the heat conduction sheet 42, and/or a heat-insulating layer 17 (see FIG. 5) may be added. Further, as with the second embodiment, the heat exchange part 41 and the heat conduction sheet 42 may be integrally covered by a covering layer 21 (see FIG. 6).

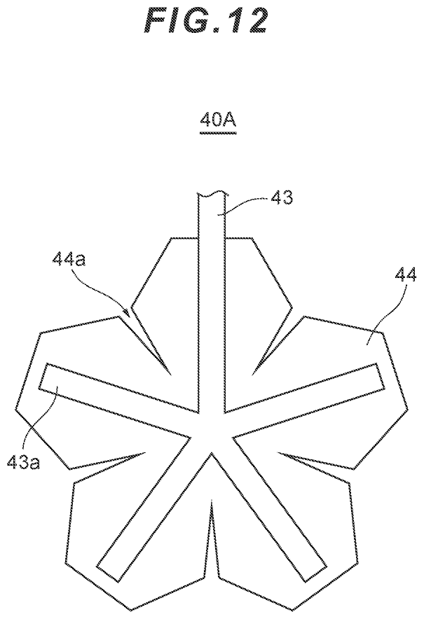

FIG. 12 is a plan view showing a variation of the cooling device according to the fourth embodiment of the present invention. The cooling device 40A shown in FIG. 12 includes: a heat exchange part 43 having a flow channel through which a refrigerant passes; and a heat conduction sheet 44 which is connected directly or indirectly to the heat exchange part 43.

The basic configuration and function of the heat exchange part 43 and the heat conduction sheet 44 in the present variation are similar to those of the heat exchange part 41 and the heat conduction sheet 42 in the above-described fourth embodiment, except that their planar shapes are different. Specifically, the heat exchange part 43 in the present variation has a plurality of tubes 43a that diverge radially. In addition, at the periphery of the heat conduction sheet 44, a plurality of notches 44a are formed that extend from the outer circumference to the center so as to avoid these tubes 43a. Thus, if the heat exchange part 43 is tubular, the arrangement of the heat exchange part 43 may be determined depending on the arrangement of the notches 44a formed in the heat conduction sheet 44. Accordingly, the heat conduction sheet 44 may be more easily closely-fitted to the site to be treated, allowing for more efficient cooling of a wide range of the site to be treated.

The present invention is not limited to the embodiments and variations described above, and may be carried out in various other forms within the scope that does not depart from the spirit of the present invention. For example, such various other forms may be formed by excluding some components from all of the components shown in the above-described embodiments and variations, or by appropriately combining the components shown in the above-described embodiments and variations.

Further advantages and modifications may be easily conceived of by those skilled in the art. Accordingly, from a wider standpoint, the present invention is not limited to the particular details and representative embodiments described herein. Accordingly, various modifications can be made without departing from the spirit or scope of the general idea of the invention defined by the appended claims and equivalents thereof.

What is claimed is:

1. An intravital cooling device for cooling a target site in a living body, comprising:
a heat exchange part having a flow channel through which a refrigerant passes; and
a flexible heat conduction sheet that is directly or indirectly connected to the heat exchange part and arranged to cover the target site, wherein the heat conduction sheet includes a living-body surface, which is a surface on the side that makes contact with the target site, and an outer surface, which is a surface opposite from the living-body surface.

2. The intravital cooling device according to claim 1, wherein a heat conductivity of the heat conduction sheet is equal to or higher than a heat conductivity of a portion of the heat exchange part that is connected to the heat conduction sheet.

3. The intravital cooling device according to claim 1, wherein the heat exchange part is arranged inwards from an outer circumference of the heat conduction sheet, and
the heat conduction sheet is connected to the heat exchange part via an intermediate layer made of an adhesive material.

4. The intravital cooling device according to claim 1, wherein the heat exchange part and the heat conduction sheet are integrated by being covered by a biocompatible covering layer.

5. The intravital cooling device according to claim 4, wherein the covering layer is formed by sputtering or vacuum deposition.

6. The intravital cooling device according to claim 4, wherein the heat exchange part is arranged on the outer surface side of the heat conduction sheet, and
a thickness of a portion of the covering layer covering the outer surface side is greater than a thickness of a portion of the covering layer covering the living-body surface side of the heat conduction sheet.

7. The intravital cooling device according to claim 1, wherein the heat conduction sheet is covered by a biocompatible coating film.

8. The intravital cooling device according to claim 1, wherein a heat-insulating layer having a heat conductivity lower than that of the heat conduction sheet is arranged on at least a portion of a region of the outer surface side of the heat exchange part and the heat conduction sheet.

9. The intravital cooling device according to claim 1, wherein a protrusion used for fixing the heat exchange part in the living body is provided to the heat exchange part.

10. The intravital cooling device according to claim 1, wherein a through-hole, a protrusion, or a notch used for fixing the heat conduction sheet in the living body is formed in a portion of the heat conduction sheet.

11. The intravital cooling device according to claim 1, wherein the heat exchange part is a member having a flow channel formed inside a solid body.

12. The intravital cooling device according to claim 1, wherein the heat exchange part is a flexible tubular body.

13. The intravital cooling device according to claim 1, wherein one or more notches are formed on a periphery of the heat conduction sheet.

14. The intravital cooling device according to claim 1, wherein the heat conduction sheet is a carbon graphite sheet or a metal foil.
